(19) United States
(12) Patent Application Publication
(10) Pub. No.: US 2004/0175404 A1
(43) Pub. Date: Sep. 9, 2004

(54) Moisture triggered sealed release system

(76) Inventors: Adi Shefer, East Brunswick, NJ (US); Samuel Shefer, East Brunswick, NJ (US)

Correspondence Address: Diane Dunn McKay, Esq., Mathews, Collins, Shepherd & McKay, P.A., Suite 306, 100 Thanet Circle, Princeton, NJ, 08540, US

(21) Appl. No.: 10/731,319

(22) Filed: Dec. 8, 2003

Related U.S. Application Data
(63) Continuation-in-part of application No. 10/122,549, filed on Apr. 15, 2002.

(51) Int. Cl. A61K 7/00

(52) U.S. Cl. 424/401

(57) ABSTRACT

The present invention relates to an improved fragrance controlled release system that can be incorporated into anhydrous consumer and cosmetic products such as powder laundry detergents, underarm deodorant or antiperspirant sticks, soap bars, body deodorant powders, foot spray, hygiene sprays, feminine napkin sprays, undergarment sprays, and the like that provides a high intensity odor signal such as a high impact fragrance burst in response to moisture. A selected fragrance is encapsulated in a water-sensitive matrix material. The high impact fragrance burst in response to moisture is achieved by formulating the fragrance or fragrance ingredients that are encapsulated to provide improved fragrance release in response to moisture. The encapsulated fragrance is formulated by combining fragrance ingredients such that their interaction with water results in increasing their relative content in the headspace of the system proximate environment after the system has been exposed to moisture. Examples of suitable fragrance ingredients have ClogP≦4.0 and boiling point≦300° C. The present invention also can provide multiple fragrance bursts in response to moisture. Multiple fragrance bursts can be achieved by formulating the moisture sensitive matrix material with materials having different dissolution rates. The invention further relates to anhydrous consumer and cosmetic product compositions comprising the improved fragrance controlled release system of the present invention.

### CROSS REFERENCE TO RELATED APPLICATION

This application is a Continuation-In-Part of U.S. patent application Ser. No. 10/122,549, filed Apr. 15, 2002, hereby incorporated in its entirety by reference into this application.

### BACKGROUND OF THE INVENTION

1. Field of the Invention

The present invention relates to an improved fragrance controlled release system that completely seals the fragrance under ambient conditions, or during the product shelf life such that no fragrance is released until the system is contacted with moisture. More particularly, the invention relates to an improved controlled release system that completely seals menthol under ambient conditions, or during the product shelf life such that no menthol is released until the system is contacted with moisture.

2. Description of the Related Art

Attempts have been made to provide fragrance triggered release systems (in response to moisture) by encapsulating the fragrance in water sensitive materials.

U.S. Pat. No. 3,971,852 discloses the use of spray-dried fragrance particles composed of starch derivatives, natural gums (e.g., gum arabic), and polyhydroxy compounds (i.e., mannitol, sorbitol) in cosmetic, personal care, and household products. U.S. Pat. No. 4,339,356 also discloses spray-dried fragrance in a water soluble polymeric matrix that emits perfume for a substantial length of time and upon contact with water emits perfume strongly. These patents do not provide any disclosure of selection of selection of types of fragrances for completely sealing the fragrance and improved release upon contact with water.

U.S. Pat. Nos. 4,803,195 and 5,508,259 disclose nonaqueous perfuming compositions intended for use in perfumed articles and devices, comprising at least two perfuming elements, wherein each of said perfuming elements has an olfactive character distinct from that of the others, and wherein one of said perfuming elements is in liquid form and the others in water-soluble microencapsulated form. The perfuming composition according to the invention makes it possible to suppress body malodors through topical application thereof on the human body skin and provides advantageous olfactive effects when used for perfuming soaps and powder detergents. By perfuming element, the invention discloses any active odoriferous ingredient or any perfuming base or mixture of two or more active odoriferous ingredients of current use in perfumery. The matrix material utilized in the above patents comprises:

a. a solid film-forming substrate chosen from polyvinyl acetate, polyvinyl alcohol, dextrins, natural or modified starch, vegetable gums, pectins, xanthans, carboxymethylcellulose, methylcellulose, hydroxymethylcellulose and lipoheteropolysaccharides, and

b. an emulsifying agent chosen from mono- or diglycerides of fatty acids, esters derived from the combination of fatty acids with sorbitol or a saccharide, or their alkoxylated derivatives, or an ester of tartaric, citric, ascorbic or lactic acid.

The use of emulsifying agents in these systems increases the solubility of the fragrance in the matrix. The increased solubility of the fragrance in the matrix may affect its permeability through the matrix (C. E. Rogers “Permeation of Gases and Vaours in Polymers”, page 25 in “Polymer Permeability” J. Comyn Ed., incorporated herein as reference):

P=D×S

Where:

P is the fragrance permeability

D is the diffusion coefficient, and

S is the solubility

The increased solubility of the fragrance in the matrix as a result of using emulsifying agents may reduce the ability of the matrix to sustain the release of the fragrance from the system during the product shelf life.

U.S. Pat. No. 5,069,231 discloses a controlled release system that provides an initial “burst” of fragrance as well as sustains fragrance release through the use of coating technology. The controlled release system disclosed consists of: (a) a core or a plurality of cores consisting of the same or different releasable functional materials dispersed within a first polymeric substance; (b) coated onto a substantial portion of the surface of the core or cores, a barrier polymer coating consisting of a second polymeric substance, the second polymeric substance having a permeability to the mass transport of functional material contained within the core or within one or more of the plurality of cores which is substantially less than the permeability of the first polymeric substance to the mass transport; and (c) coated onto a substantial portion of the outer surface of the barrier polymer coating, a functional material-burst coating consisting of one or more of the same or different functional material dispersed within a third polymeric substance

U.S. Pat. Nos. 6,045,835 and 6,106,875 disclose a method of encapsulating flavors and fragrances by controlled water transport into microcapsules. A method of encapsulating an amphiphilic volatile flavor or fragrance compound into a microcapsule having a hydrogel shell and an oil core. The flavor or fragrance compound in a fluid is transported into and solubilized in the core by partition coefficient equilibrium using water in the capsule wall to transport the compound into the core. The fragrances or the flavors are released in response to pressure upon capsule fracture

U.S. Pat. No. 6,235,274 discloses microparticles which controllably release olfactorily active substances using extrusion technology. Described are flavor composition, flavor component, perfume composition and perfume component-containing microparticles which are particulate matrices composed of: (a) an olfactorily active component (e.g., perfume component); (b) silica; and (c) a saccharide composition which is a mixture of mannitol and maltose. The microparticles are useful in augmenting, enhancing and/or imparting aroma and/or taste (over relatively long periods of time in a controllably releasable manner) to perfume compositions, perfumed articles (e.g., deodorancy and antiperspirant sticks), foodstuffs, chewing gums, beverages and the like. Also described is a process for preparing the above-mentioned microparticles using, in sequence, (1) adsorption of the olfactorily active material onto silica followed by (2) a blending/extrusion step followed by (3) at least one particularization step.

The prior art of which applicant is aware does not set forth a controlled release system comprising selected fragrances or fragrance ingredients that completely seals the fragrances such that no odor is released until contact with moisture and provides a high impact fragrance burst in response to moisture based on the fragrance ingredient's chemical and physical properties.

### SUMMARY OF THE INVENTION

The present invention addresses the ongoing need for fragrance controlled release systems for consumer and cosmetic products that convey to the consumer the product performance by releasing a high intensity odor signal. No engineered coordination of the utilization of the variables concerned has been shown in the prior art whereby, the fragrance ingredients that are utilized for encapsulation in moisture triggered release systems actually have the ability to provide high impact fragrance signal or burst in response to moisture (increase the fraction of the fragrance or fragrance ingredients in the headspace of the system proximate environment after the system has been exposed to moisture). Taking into account that the perception of some fragrance ingredients decreases when exposed to moisture, the careful selection of aroma chemicals to be encapsulated in moisture triggered release systems is critical to effectively deliver a high impact perceivable signal or fragrance burst.

The present invention meets the aforementioned needs in that it has been surprisingly discovered that in order for the moisture triggered release systems to provide high impact fragrance burst it is essential to formulate the fragrance that is encapsulated with fragrance ingredients that have high water solubility and/or high volatility. Suitable fragrance ingredients for encapsulation in the controlled release system of the present invention are naturally, or synthetically, derived fragrance ingredients which have high water solubility as determined by a calculated log₁₀P, or ClogP≦about 4.0, P being the n-octanol-water partition coefficient of the fragrance ingredient. The fragrance ingredients can also have high volatility being determined by a boiling point of ≦about 300° C. The fragrance ingredients solubility in water was found to have a more pronounced effect on their ability to provide a high impact fragrance burst, than the ingredients volatility. A preferred fragrance ingredient is menthol.

The term “fragrance burst” as used herein refers to release of fragrance resulting from release of fragrance ingredients from a moisture-sensitive matrix material upon exposure to water. The term “high impact fragrance burst” as used herein refers to having a high intensity of fragrance immediately released upon exposure of the delivery system to moisture. The “high impact fragrance burst” provides a level of fragrance ingredients in the headspace of the system proximate environment which is much higher than if the encapsulated fragrance comprised fragrance ingredients that have low water solubility and low volatility (ClogP>4.0 and boiling point>300° C.). It is believed that fragrance ingredients that have high water solubility and high volatility are more likely to partition or becomes associated with the water through electrostatic interactions and are carried by water vapor into the headspace.

The invention also provides a moisture activated controlled release system intended for use in consumer and cosmetic products, such as anhydrous products, that prevent the release of the fragrance during the product shelf life and release the fragrance upon need in response to moisture to provide a high impact fragrance burst.

The invention also provides a free-flowing powder comprising micro-spheres composed of water sensitive materials that encapsulate fragrance ingredients that have the desired chemical and physical properties and other active ingredients characterized by:

(i) protection of the fragrance and the active agents during storage, until needed;

(ii) moisture triggered release of the active agents upon need in response to moisture; and

(iii) high impact fragrance burst in response to moisture that “signal” the consumer that the product has been activated.

The invention further provides anhydrous consumer and cosmetic products such as deodorant or antiperspirant sticks, deodorant or antiperspirant roll on devices, deodorant or antiperspirant smooth-ons, deodorant or antiperspirant aerosols, body deodorant powders, soap bars, foot spray, hygiene sprays, feminine napkin sprays, undergarment sprays, and the like comprising the controlled release system of the present invention.

The invention will be more fully described by reference to the following drawings.

### BRIEF DESCRIPTION OF THE DRAWINGS

FIG. 1 is a scanning electron microscopy (SEM) with magnification of 5000 times of the micro-spheres of the present invention. The micro-spheres have an average particle size of 10 microns to 20 microns and a smooth surface.

FIG. 2 is a scanning electron microscopy (SEM) with magnification of 5000 times of the cross section of the micro-spheres of the present invention.

FIG. 3 is a graph of the effect of moisture on the content of fragrance ingredients in the headspace.

FIG. 4 is a graph of the effect of moisture on the content of fragrance ingredients in the headspace.

FIG. 5 is a graph of the effect of moisture on the content of fragrance ingredients that have ClopP≦4.0 in the headspace.

### DETAILED DESCRIPTION

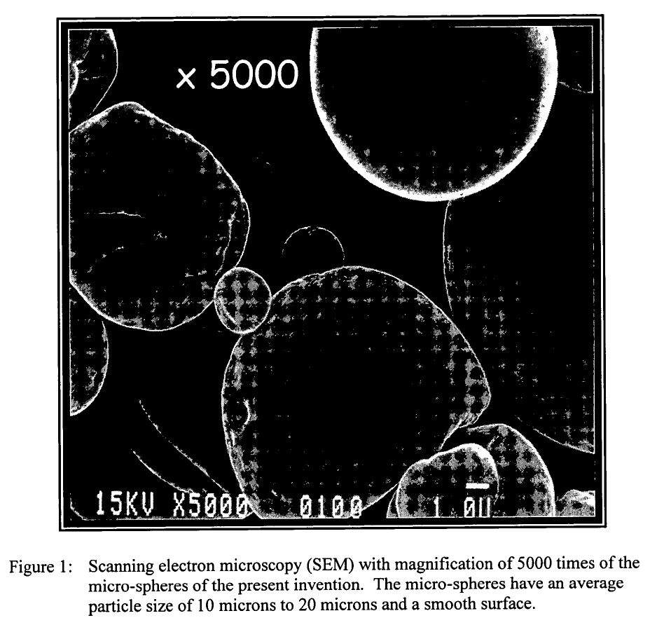

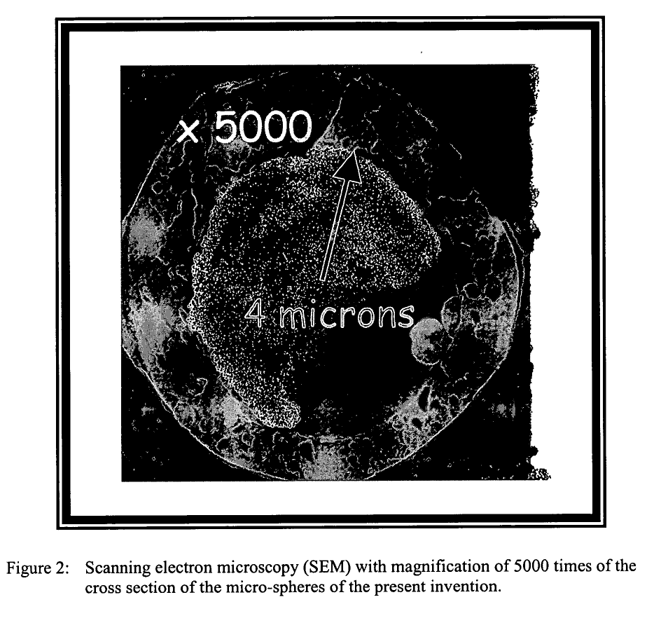

The present invention relates to an improved fragrance controlled release system that can be incorporated into a consumer and cosmetic products such as and anhydrous consumer or cosmetic product to provide high intensity odor signal of a high impact fragrance burst in response to moisture. Examples of anhydrous consumer or cosmetic products include underarm deodorant or antiperspirant sticks, soap bars, body deodorant powders, foot spray, hygiene sprays, feminine napkin sprays, undergarment sprays, and the like. The controlled delivery system of the present invention is a free-flowing powder in the form of microspheres composed of water sensitive materials that either dissolves or swells in response to moisture, as shown in FIG. 1 and FIG. 2. The micro-spheres of the present invention have smooth surface (FIG. 1) and a porous wall (FIG. 2). The term “spheres” is intended to describe solid, substantially spherical particulates. It is appreciated that other particle shapes can be included in the term “sphere” in accordance with the teachings of the present invention.

The fragrance encapsulated in the controlled delivery system of the present invention is formulated by combining fragrance ingredients that have defined chemical and physical properties, such as high water solubility and high volatility (ClogP≦4.0 and boiling point<300° C.) in order to maximize the level of fragrance ingredients in the system proximate environment headspace after the system has been exposed to moisture thereby providing a high odor intensity signal. Suitable encapsulated fragrance ingredients of the present invention are naturally, or synthetically, derived fragrance ingredients which have high water solubility as determined by a calculated log₁₀P, or ClogP≦4.0, P being the n-octanol-water partition coefficient of the fragrance ingredient and high volatility being determined by a boiling point<300° C. It will be appreciated that other chemical or physical properties known to those skilled in the art can be used to determine high water solubility and high volatility.

Additional active ingredients can be added to the controlled release system of the present invention including, but are not limited to: anti-oxidants; free radical scavengers; moisturizers; depigmentation agents; reflectants; humectants; anti-microbial agents, antibacterial agents; allergy inhibitors; anti-acne agents; anti-aging agents; anti-wrinkling agents, antiseptics; analgesics; keratolytic agents; anti-inflammatory agents; fresheners; healing agents; anti infectives; inflammation inhibitors; wound healing promoters; peptides, polypeptides and proteins; deodorants and antiperspirants; skin emollients and skin moisturizers; tanning agents; skin lightening agents; anti-fungal; depilating agents; counterirritants; poison ivy products; poison oak products; burn products; make-up preparations; vitamins; amino acids and their derivatives; herbal extracts; sensory markers; cooling agents; heating agents; skin conditioners; chelating agents; cell turnover enhancers; coloring agents; sunscreens; nourishing agents; moisture absorbers; sebum absorbers and the like; skin penetration enhancers; and other active ingredients. The additional components can be present in an amount of from about 0% to about 20% by weight of the micro-spheres.

Selection of fragrance Ingredients for Encapsulation

Fragrances useful for the present invention can be a single aroma chemical, a fragrance accord relatively simple in composition, or can comprise highly sophisticated, complex mixtures of natural and synthetic chemical components, all chosen to provide any desired odor. The encapsulated single aroma chemical, fragrance accord, or full fragrance can be the same or different than a fragrance that is being used as neat oil. The encapsulated fragrance ingredients are preferably selected from the fragrance ingredients comprising the neat fragrance. The encapsulated fragrance is formulated by combining fragrance ingredients that have high water solubility and high volatility. Suitable fragrance ingredients ClogP≦4.0 and/or a boiling point≦300° C. to maximize the level of fragrance ingredients in the system proximate environment headspace after the system has been exposed to moisture to provide a high odor intensity signal. In an embodiment of the present invention, the fragrance ingredient is menthol.

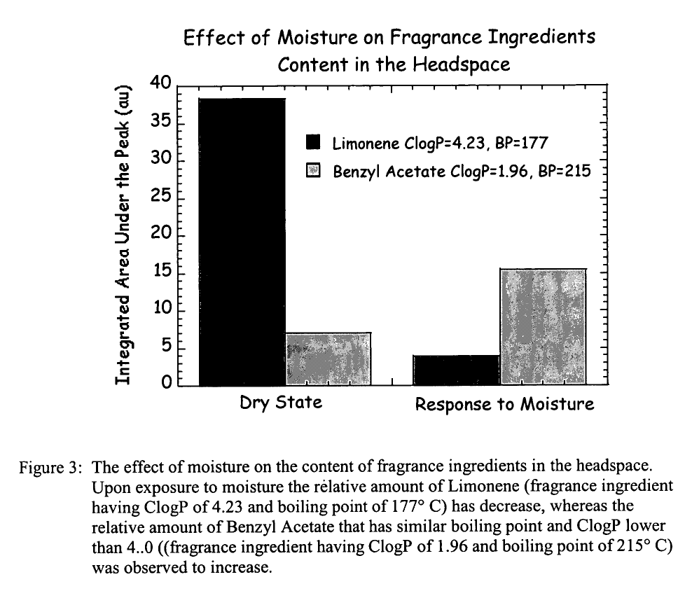

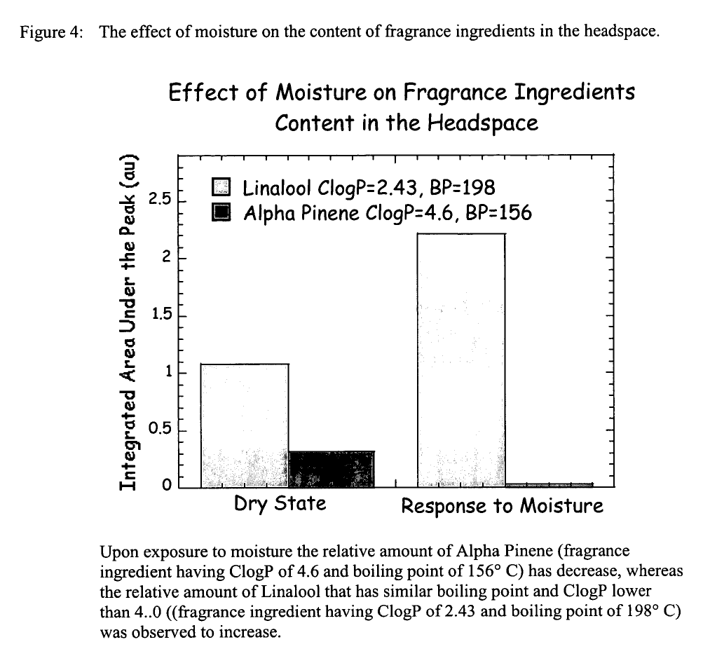

It has been unexpectedly found that the relative amount of fragrance ingredients in the headspace after exposure to water is closely dependent on the ingredient water solubility (ClogP) and/or volatility. The term “headspace” as used herein refers to sampling of a vapor phase above and around the sample. Headspace sampling or headspace analysis ensures that only volatile materials that are delivered to the vapor phase are analyzed. The solubility in water, or the ability to partition into the aqueous phase, was found to dominate the ability of fragrance ingredients to partition in to the headspace when exposed to water. Fragrance volatility was also observed to affect the ability of fragrance ingredients to partition into the headspace to a lesser extent than the water solubility (ClogP). FIGS. 3 and 4 illustrate the effect of moisture on different fragrance ingredients. The fragrance ingredient of limonene, having ClogP of 4.23 and boiling point of 177° C. and the fragrance and Benzyl Acetate having ClogP of 1.96 and a boiling point of 215° C. were measured in the dry state and in response to moisture, as shown in FIG. 3. The term “au” as used herein refers to arbitrary units. A fragrance ingredient, alpha pinene, having ClogP of 4.6 and boiling point of 156° C. and linalool having ClogP of 2.43 and boiling of 198° C. were measured in the dry state and in response to moisture, as shown in FIG. 4. Upon exposure to moisture the relative amount of fragrance ingredients that have ClogP>4.0 in the headspace was observed to decrease as compare to fragrance ingredients that have ClogP<3.0, although their boiling point was similar in value. Accordingly, when a fragrance is composed primarily of ingredients having a ClogP of about 4.0, or lower and a boiling point of 300° C. or lower, the fragrance responds to moisture by increasing its relative amount in the headspace to provide a high impact fragrance burst that can be easily perceived by the consumer. Menthol, another fragrance ingredient, that has ClogP of 3.43 and a boiling point of 216° C. was measured in the dry state and in response to moisture. Upon exposure to moisture the relative amount of menthol in the headspace was observed to increase and provided a high impact fragrance burst that can be easily perceived by the consumer.

The boiling points of many perfume ingredients are given in, e.g., “Perfume and Flavor Chemicals (Aroma Chemicals),” S. Arctander, published by the author, 1969, incorporated herein by reference. Other boiling point values can be obtained from different chemistry handbooks and databases, such as the Beilstein Handbook, Lange's Handbook of Chemistry, and the CRC Handbook of Chemistry and Physics. When a boiling point is given only at a different pressure, usually lower pressure than the normal pressure of 760 mm Hg, the boiling point at normal pressure can be approximately estimated by using boiling point-pressure nomographs, such as those given in “The Chemist's Companion,” A. J. Gordon and R. A. Ford, John Wiley & Sons Publishers, 1972, pp. 30-36. When applicable, the boiling point values can also be calculated by computer programs, based on molecular structural data, such as those described in “Computer-Assisted Prediction of Normal Boiling Points of Pyrans and Pyrroles”, D. T. Stanton et al, J. Chem. Inf. Comput. Sci., 32 (1992), pp. 306-316, “Computer-Assisted Prediction of Normal Boiling Points of Furans, Tetrahydrofurans, and Thiophenes”, D. T. Stanton et al, J. Chem. Inf. Comput. Sci., 31 (1992), pp. 301-310, and references cited therein, and “Predicting Physical Properties from Molecular Structure,” R. Murugan et al, Chemtech, June 1994, pp. 17-23. All the above publications are incorporated herein by reference.

The ClogP of many perfume ingredients has been reported; for example, the Pomona92 database, available from Daylight Chemical Information Systems, Inc. (Daylight CIS), Irvine, Calif., contains many, along with citations to the original literature. However the logP values are most conveniently calculated by the “CLOGP” program, also available from Daylight CIS. This program also lists experimental logP values when they are available in the Pomona92 database. The “calculated logP” (ClogP) is determined by the fragment approach of Hansch and Leo (cf., A. Leo, in Comprehensive Medicinal Chemistry, Volume 4, C. Hansch, P. G. Sammens, J. B. Taylor and C. A. Ramsden, editors, page 295, Pergamon Press, 1990, incorporated by reference herein). The fragment approach is based on the chemical structure of each perfume ingredient and takes into account the numbers and types of atoms, the atom connectivity and the chemical bonding. The ClogP values, which are the most reliable and widely used estimates for this physiochemical property, are preferably used instead of the experimental logP values in the selection of fragrance ingredients which are useful in the present invention.

Fragrance ingredients that are encapsulated in the controlled release system of the present invention to provide high impact fragrance burst in response to moisture are those having a having a ClogP of less than about 4.0, or ClogP of less than about 4.0 and boiling point lower than about 300° C. Table 1 provides nonlimiting examples of fragrance ingredients that can be incorporated in the controlled release system of the present invention to provide a high impact fragrance burst in response to moisture.

TABLE 1
Examples of bursting fragrance ingredients (ClogP ≦ 4.0
and boiling point ≦ 300° C.) that can be used in the
controlled release system of the present invention

Approximate

| Aroma Chemical | Boiling Points | ClogP |
|---|---|---|
| Menthol | 216 | 3.43 |
| Phenyl ethyl alcohol | 220 | 1.18 |
| Coumarin | 291 | 1.41 |
| Benzaldehyde | 179 | 1.48 |
| Hydroxycitronellal | 241 | 1.54 |
| Vanillin | 285 | 1.58 |
| Benzyl acetate | 215 | 1.96 |
| laevo-Carvone | 231 | 2.08 |
| Indole | 254 | 2.14 |
| beta-Methyl naphthyl ketone | 300 | 2.28 |
| Eugenol | 253 | 2.31 |
| Linalool | 198 | 2.43 |
| iso-Eugenol | 266 | 2.55 |
| alpha-Terpineol | 219 | 2.57 |
| Methyl cinnamate | 263 | 2.62 |
| Geraniol | 230 | 2.65 |
| Nerol | 227 | 2.65 |
| cis-Jasmone | 248 | 2.71 |
| Geranyl acetate | 245 | 3.72 |
| delta-Nonalactone | 280 | 2.76 |
| Methyl-N-methyl anthranilate | 256 | 2.79 |
| Dihydro myrcenol | 208 | 3.03 |
| Benzophenone | 306 | 3.12 |
| alpha-Citronellol | 225 | 3.19 |
| Yara-yara | 274 | 3.24 |
| 2-Methoxy naphthalene | 274 | 3.24 |
| Phenyl hexanol | 258 | 3.30 |
| Amyl benzoate | 262 | 3.42 |
| Phenyl heptanol | 261 | 3.48 |
| iso-Bornyl acetate | 227 | 3.49 |
| Linalyl acetate | 220 | 3.50 |
| delta-Undecalactone | 290 | 3.83 |
| Lilial | 258 | 3.86 |
| Allyl cyclohexane propionate | 267 | 3.93 |
| and mixtures thereof. | | |

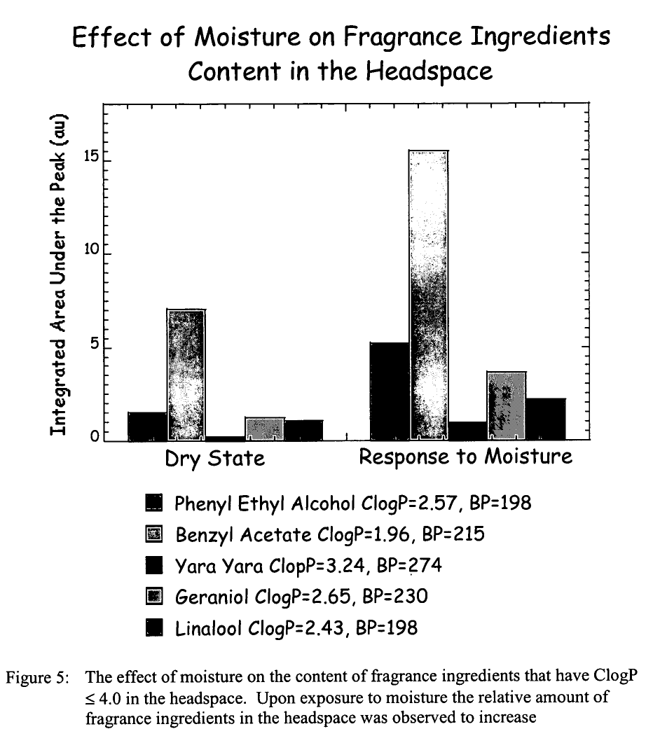

FIG. 5 illustrates the ability of fragrance ingredients having chemical and physical properties of ClogP≦4.0 and boiling point≦300° C. to provide a burst in response to moisture.

In alternate embodiments of compositions, some fragrance ingredients that have ClogP>about 4.0 and boiling point>about 300° C. can be used in small amounts, such as to improve product odor. In order to ensure high odor intensity upon exposure of the system to moisture, the fragrance compositions of the present invention contain less than about 30 weight percent of nonbursting fragrance ingredients (fragrance ingredients that have ClogP>about 4.0 and boiling point>about 300° C.), preferably less than about 25 weight percent of nonbursting fragrance ingredients, more preferably, less than about 20 weight percent of nonbursting fragrance ingredients, and even more preferably, less than about 15 weight percent of nonbursting fragrance ingredients.

Water Sensitive Matrix Materials and Process of Micro-Sphere Formation

Moisture-sensitive materials for forming the micro-spheres of the present invention comprise of water soluble and water dispersible synthetic and natural polymers and copolymers, such as polyvinyl alcohol, starch derivatives, polysaccharides, hydrocolloids, natural gums, proteins, and mixtures thereof. The moisture-sensitive materials can be activated by water or body fluids. The term “body fluids”, as used herein, includes eccrine sweat, apocrine sweat, sebum, build up of sensible moisture from transepidermal water loss, vaginal discharge, urine, and mixtures thereof.

Examples of other synthetic water sensitive polymers which are useful for forming the moisture sensitive of the present invention include polyvinyl pyrrolidone, water soluble celluloses, ethylene maleic anhydride copolymer, methylvinyl ether maleic anhydride copolymer, acrylic acid copolymers, anionic polymers of methacrylic acid and methacrylate, cationic polymers with dimethyl-aminoethyl ammonium functional groups, polyethylene oxides, water soluble polyamide or polyester.

Examples of water soluble hydroxyalkyl and carboxyalkyl celluloses include hydroxyethyl and carboxymethyl cellulose, hydroxyethyl and carboxyethyl cellulose, hydroxymethyl and carboxymethyl cellulose, hydroxypropyl carboxymethyl cellulose, hydroxypropyl methyl carboxyethyl cellulose, hydroxypropyl carboxypropyl cellulose, hydroxybutyl carboxymethyl cellulose, and the like. Also useful are alkali metal salts of these carboxyalkyl celluloses, particularly and preferably the sodium and potassium derivatives.

The polyvinyl alcohol useful in the practice of the invention is partially and fully hydrolyzed polyvinyl acetate, termed “polyvinyl alcohol” with polyvinyl acetate as hydrolyzed to an extent also termed degree of hydrolysis, of from about 75% up to about 99%. Such materials are prepared by means of any of Examples I-XIV of U.S. Pat. No. 5,051,222 issued on Sep. 24, 1991, the specification for which is incorporated by reference herein.

The dissolution rate of polyvinyl alcohol in water is significantly slower than that of maltodextrins and polysaccharide. The dissolution rate of polyvinyl alcohol in water is strongly affected by the polymer degree of hydrolysis and molecular weight. Accordingly, polyvinyl alcohol can be utilized in the present invention to delay the response of the system to moisture (affect the kinetic of response to moisture), as well as be combined with materials having a faster dissolution rate in order to provide multiple bursts in response to moisture. Polyvinyl alcohol useful for practice of the present invention is Mowiol® 3-83, having a molecular weight of about 14,000 Da and degree of hydrolysis of about 83%, Mowiol® 3-98 and a fully hydrolyzed (98%) polyvinyl alcohol having a molecular weight of 16,000 Da commercially available from Gehring-Montgomery, Inc. of Warminister Pa. Other suitable polyvinyl alcohols are: AIRVOL® 205, having a molecular weight of about 15,000-27,000 Da and degree of hydrolysis of about 88%, and VINEX® 1025, having molecular weight of 15,000-27,000 Da degree of hydrolysis of about 99% and commercially available from Air Products & Chemicals, Inc. of Allentown, Pa.; ELVANOL® 51-05, having a molecular weight of about 22,000-26,000 Da and degree of hydrolysis of about 89% and commercially available from the DuPont Company, Polymer Products Department, Wilmington, Del.; ALCOTEX® 78 having a degree of hydrolysis of about 76% to about 79%, ALCOTEX® F88/4 having a degree of hydrolysis of about 86% to about 88% and commercially available from the Harlow Chemical Co. Ltd. Of Templefields, Harlow, Essex, England CM20 2BH; and GOHSENOL® GL-03 and GOHSENOL® KA-20 commercially available from Nippon Gohsei K.K., The Nippon Synthetic Chemical Industry Co., Ltd., of No. 9-6, Nozaki Cho, Kita-Ku, Osaka, 530 Japan.

Suitable polysaccharides are polysaccharides of the nonsweet, coloidally-soluble types, such as natural gums, for example, gum arabic, starch derivates, dextrinized and hydrolyzed starches, and the like. A suitable polysaccharide is a water dispersible, modified starch commercially available as Capul®, N-Lok®, Hi-Cap™ 100 or Hi-Cap™ 200 commercially available from the National Starch and Chemical Company of Bridgewater, N.J.; Pure-Cote™, commercially available from the Grain Processing Corporation of Muscatine, Iowa. In the preferred embodiment, the natural gum is a gum arabic, commercially available from TIC Gums Inc. Belcamp, Midland. Suitable hydrocolloids are xanthan, maltodextrin, galactomanan or tragacanth, preferably maltodextrins such as Maltrin™ M100, and Maltrin™ M150, commercially available from the Grain Processing Corporation of Muscatine, Iowa.

The amount of fragrance based in moisture sensitive micro-sphere can be in the range of from about 1% up to about 60% by weight of the moisture sensitive micro-sphere.

The micro-spheres of the present invention can be prepared by the steps of (1) forming an aqueous phase of the moisture sensitive materials (either a single material or mixture of several materials); (2) emulsifying the fragrance, fragrance accord, or fragrance ingredient and other active agents in the aqueous phase; and (3) removing moisture to create free-flowing powder. For example, moisture can be removed by spray drying droplets of emulsion. Spray drying is well known in the art and been used commercially in many applications, including foods where the core material is a flavoring oil and cosmetics where the core material is a fragrance oil, as described in Cf. Balassa, “Microencapsulation in the Food Industry”, CRC Critical Review Journal in Food Technology, July 1971, pp 245-265; Barreto, “Spray Dried Perfumes for Specialties, Soap and Chemical Specialties”, December 1966; Maleeny, Spray Dried Perfumes, Soap and San Chem, January 1958, pp. 135 et seq.; Flinn and Nack, “Advances in Microencapsulation Techniques”, Batelle Technical Review, Vo. 16, No. 2, pp. 2-8 (1967); U.S. Pat. No. 5,525,367; and 5,417,153 which are incorporated herein as references.

In one embodiment, micro-spheres that have the ability to provide multiple fragrance bursts in response to moisture are formed by mixing the fragrance and a selected active agent with polyvinyl alcohol, or compositions of polyvinyl alcohol and polysaccharides, under conditions sufficient to encapsulate the fragrance. Preferably mixing a selected fragrance and active agent with the polyvinyl alcohol, or compositions of polyvinyl alcohol and polysaccharides, until the emulsion is formed and then spray drying the emulsion to thereby form an encapsulated micro-sphere. In the preferred embodiment, the moisture sensitive matrix is formed of a polyvinyl alcohol material at a level of from about 1% to about 80%, preferably from about 1% to about 70% by weight of the matrix material with the balance being the amount by weight of active agents and an optimal amount of polysaccharides. In an alternate embodiment, polyvinyl alcohol is present in the matrix material in an amount of about 1% to about 80% by weight and polysaccharides are present in the matrix material in an amount of about 1% to about 80% by weight. In the preferred embodiment, the fragrance ingredients are generally present at a level from about 0.01% to about 80% preferably from about 1% to about 50% by weight of the encapsulated active agent with the balance being the polyvinyl alcohol or polyvinyl alcohol and polysaccharides. Optionally other conventional ingredients known in the art such as preservatives can be used in accordance with the teachings of the present invention. The micro-spheres of the present invention preferably have size of from about 0.5 micron to about 300 microns, more preferably from about 1 micron to about 200 microns, most preferably from about 2 microns to about 30 microns. The present invention preferably has minimal active agents on the surface of the spheres, preferably less than 1%.

The invention can be further illustrated by the following examples thereof, although it will be understood that these examples are included merely for purposes of illustration and are not intended to limit the scope of the invention unless otherwise specifically indicated. All percentages, ratios, and parts herein, in the Specification, Examples, and claims, are by weight and are approximations unless otherwise stated.

### The Neat Fragrance Oil

### EXAMPLE 1

The composition of the fragrance that was used as neat oil is detailed to the following table. The neat fragrance oil is composed of a wide range of fragrance ingredients including both bursting and nonbursting ingredients.

| | | | Boiling Point |
|---|---|---|---|
| Fragrance Composition | Component (% Wt.) | ClogP | (° C.) |
| Alpha Pinene | 15.0 | 4.6 | 198 |
| Geraniol | 5.0 | 2.65 | 230 |
| Dihydro Myrcenol | 5.0 | 3.03 | 208 |
| Linalool | 5.0 | 2.43 | 198 |
| Lemonene | 20.0 | 4.23 | 177 |
| alpha-Citronellol | 5.0 | 3.19 | 225 |
| Phenyl Ethyl Alcohol | 5.0 | 2.57 | 219 |
| Linalyl benzoate | 10.0 | 5.233 | 263 |
| Galaxolide | 30.0 | 5.482 | +250 |

### Creation of a Fragrance Accord for Encapsulation

### EXAMPLE 2

The fragrance ingredients for encapsulation have ClogP≦4.0 and boiling point≦300° C. and are selected from the composition of the neat fragrance.

| | | | Boiling Point |
|---|---|---|---|
| Fragrance Composition | Component (% Wt.) | ClogP | (° C.) |
| Geraniol | 30.0 | 2.65 | 230 |
| Dihydro Myrcenol | 20.0 | 3.03 | 208 |
| Linalool | 15.0 | 2.43 | 198 |
| alpha-Citronellol | 5.0 | 3.19 | 225 |
| Phenyl Ethyl Alcohol | 30.0 | 2.57 | 219 |

### Preparation of Dual Fragrance Controlled Release System

### EXAMPLE 3

The following procedure is used for the preparation of a controlled release system that provides high impact fragrance burst in response to moisture. The micro-sphere water sensitive matrix is a mixture of Hi-Cap™ 100 (commercially available from the National Starch and Chemical Company of Bridgewater, N.J.), modified starch, Pure-Cote™, commercially available from the Grain Processing Corporation of Muscatine, Iowa, and maltodextrin, Maltrin™ M100, commercially available from the Grain Processing Corporation of Muscatine, Iowa.

400 grams of the fragrance accord of Example 2 are emulsified with 240 grams of Hi-Cap™ 100 (commercially available from the National Starch and Chemical Company of Bridgewater, N.J.), 100 grams of Pure-Cote™ (commercially available from the Grain Processing Corporation of Muscatine, Iowa), 60 grams of Maltrin™ M100 (commercially available from the Grain Processing Corporation of Muscatine, Iowa), and 1200 grams of water. The resulting emulsion is spray dried with a Bowen Lab Model Drier (at Spray-Tek of Middlesex, N.J.) utilizing 250 c.f.m of air with an inlet temperature of 380° F., and outlet temperature of 225° F. and a wheel speed of 45,000 r.p.m to produce a free flowing, dry powder, consisting of 50% encapsulated fragrance.

### EXAMPLE 4

400 grams of the fragrance accord of Example 1 are emulsified with 240 grams of Hi-Cap™ 100 (commercially available from the National Starch and Chemical Company of Bridgewater, N.J.), 100 grams of Pure-Cote™ (commercially available from the Grain Processing Corporation of Muscatine, Iowa), 60 grams of Maltrin™ M100 (commercially available from the Grain Processing Corporation of Muscatine, Iowa), and 1200 grams of water. The resulting emulsion is spray dried with a Bowen Lab Model Drier (at Spray-Tek of Middlesex, N.J.) utilizing 250 c.f.m of air with an inlet temperature of 380° F., and outlet temperature of 225° F. and a wheel speed of 45,000 r.p.m to produce a free flowing, dry powder, consisting of 50% encapsulated fragrance.

### EXAMPLE 5

The following procedure is used for the preparation of a controlled release system that provides high impact fragrance burst in response to moisture. The micro-sphere water sensitive matrix is polyvinyl alcohol a having a molecular weight of about 14,000 Da and degree of hydrolysis of about 83% (Mowiol® 3-83, trademark and commercially available from Gehring-Montgomery, Inc. of Warminster Pa.).

200 grams of the fragrance accord of Example 2 are emulsified with 400 grams polyvinyl alcohol a having a molecular weight of about 14,000 Da and degree of hydrolysis of about 83% (Mowiol® 3-83, trademark and commercially available from Gehring-Montgomery, Inc. of Warminster Pa.) and 1400 grams of water. The resulting emulsion is spray dried with a Bowen Lab Model Drier (at Spray-Tek of Middlesex, N.J.) utilizing 250 c.f.m of air with an inlet temperature of 380° F., and outlet temperature of 225° F. and a wheel speed of 45,000 r.p.m to produce a free flowing, dry powder, consisting of 30% encapsulated fragrance.

### EXAMPLE 6

200 grams of the fragrance accord of Example 1 are emulsified with 400 grams polyvinyl alcohol a having a molecular weight of about 14,000 Da and degree of hydrolysis of about 83% (Mowiol® 3-83, trademark and commercially available from Gehring-Montgomery, Inc. of Warminster Pa.) and 1400 grams of water. The resulting emulsion is spray dried with a Bowen Lab Model Drier (at Spray-Tek of Middlesex, N.J.) utilizing 250 c.f.m of air with an inlet temperature of 380° F., and outlet temperature of 225° F. and a wheel speed of 45,000 r.p.m to produce a free flowing, dry powder, consisting of 30% encapsulated fragrance.

### EXAMPLE 7

The following procedure is used for the preparation of a controlled release system that provides high impact fragrance burst in response to moisture. The micro-sphere water sensitive matrix is a mixture of polyvinyl alcohol a having a molecular weight of about 14,000 Da and degree of hydrolysis of about 83% (Mowiol® 3-83, trademark and commercially available from Gehring-Montgomery, Inc. of Warminster Pa.) and Hi-Cap™ 200 (commercially available from the National Starch and Chemical Company of Bridgewater, N.J.).

200 grams of the fragrance accord of Example 2 are emulsified with 20 grams polyvinyl alcohol a having a molecular weight of about 14,000 Da and degree of hydrolysis of about 83% (Mowiol® 3-83, trademark and commercially available from Gehring-Montgomery, Inc. of Warminster Pa.), 360 grams of Hi-Cap™ 200 (commercially available from the National Starch and Chemical Company of Bridgewater, N.J.), and 1200 grams of water. The resulting emulsion is spray dried with a Bowen Lab Model Drier (at Spray-Tek of Middlesex, N.J.) utilizing 250 c.f.m of air with an inlet temperature of 380° F., and outlet temperature of 225° F. and a wheel speed of 45,000 r.p.m to produce a free flowing, dry powder, consisting of 50% encapsulated fragrance.

### EXAMPLE 8

200 grams of menthol is emulsified with 800 grams polyvinyl alcohol a having a molecular weight of about 14,000 Da and degree of hydrolysis of about 83% (Mowiol® 3-83, trademark and commercially available from Gehring-Montgomery, Inc. of Warminster Pa.) and 1500 grams of water. The resulting emulsion is spray dried with a Bowen Lab Model Drier (at Spray-Tek of Middlesex, N.J.) utilizing 250 c.f.m of air with an inlet temperature of 380° F., and outlet temperature of 225° F. and a wheel speed of 45,000 r.p.m to produce a free flowing, dry powder, consisting of 20% encapsulated menthol.

### Incorporation of the Controlled Release System in Underarm Products

### EXAMPLE 9

The performance of an antiperspirant product comprising the fragrance controlled release system of Examples 3 to Example 8 (i.e., the ability to yield a high impact multiple fragrance “burst” upon perspiration) was evaluated using 0.5% neat fragrance oil of Example 1 and 1.5% encapsulated fragrance (encapsulated nonbursting fragrance (Example 1) or encapsulation of bursting fragrance (Example 2)). The un-fragranced antiperspirant base was a commercial Degree® unscented antiperspirant stick available from Helene Curtis Company of Chicago, Ill. that is fragrance free.

The control samples were prepared by weighting into a jar the appropriate amount of the neat fragrance of example 1 or the encapsulated nonbursting fragrances and the appropriate amount of the unscented Degree® base and the resulting mixture was melted at 80° C. and mixed for about one minute. The melt was poured into an underarm container and allowed to cool for an hour. The antiperspirant stick comprising the controlled release system of the present invention is prepared by weighting the appropriate amount of the neat fragrance of Example 1 the appropriate amount of the encapsulated fragrance powder and the unscented Degree® base melt into a jar. The melt was poured into an underarm container and allowed to cool for an hour.

The antiperspirant samples were applied on the forearm. The ability of the products to provide multiple fragrance burst was evaluated one hour after application of the product by misting the area with water. The products comprising the controlled release system of the present invention were found to provide high impact fragrance burst upon wetting the area whereas the control sample comprising the neat oil did not. The forearm areas treated with the antiperspirant comprising the neat fragrance (control) and that treated with the antiperspirant comprising the encapsulated fragrance were evaluated for their ability to provide high impact burst again after 6 hours (provide multiple fragrance burst). The ability of the systems to provide high impact fragrance burst was determined by olfactive evaluation. Odor perception is, by its nature, a very subjective determination. According to the procedure, the samples to be tested are provided to a panel of six odor specialists who independently rank the odor intensity on a scale of 1 (least) to 10 (most) for odor and intensity. Samples yielding an odor ranking below about 3.0 possess an odor which would hardly be noticed by the general public. The odor evaluation results were as follow:

| | Response to Moisture | |
|---|---|---|
| | Evaluated after | Evaluated after |
| | 1 Hour | 6 Hours |
| Neat Fragrance Oil (Example 1) | 4 | 2 |
| Encapsulated Fragrance (Example 3) | 7 | 5 |
| Encapsulated Fragrance (Example 4) | 4 | 3 |
| Encapsulated Fragrance (Example 5) | 6 | 6 |
| Encapsulated Fragrance (Example 6) | 3 | 4 |
| Encapsulated Fragrance (Example 7) | 8 | 5 |
| Encapsulated Fragrance (Example 8) | 4 | 4 |

These results clearly show that only the forearm treated with the encapsulated bursting fragrance of Example 2 comprising fragrance ingredients with ClogP≦4.0 and boiling point≦300° C. provide multiple high impact fragrance burst that can be easily perceived. The forearm treated with the neat fragrance or the encapsulated nonbursting fragrance of Example 1 had very low intensity signal in response to moisture.

### Incorporation of the Controlled Release System in Body Deodorant Powder

### EXAMPLE 10

The performance of a deodorant body powder comprising the fragrance controlled release system of Example 3-8 (i.e., the ability to yield a high impact fragrance “burst” upon perspiration) was evaluated and compared to the performance of the same product comprising the neat fragrance, at the same fragrance level. The un-fragranced body deodorant powder base was unscented Pure-Dent® commercially available from Grain Processing Corporation of Muscatine, Iowa.

The body deodorant powders were prepared at a 1% effective fragrance concentration using the fragrance described in Example 1 and Example 2. The control samples was prepared by weighting into a jar appropriate amounts of the neat fragrance or encapsulated fragrance of Example 4, Example 6, and Example 8 appropriate amounts of the unscented Pure-Dent® powder followed by mixing the powder well. The body deodorant powder comprising the controlled release system of the present invention is prepared by weighting appropriate amounts of the unscented Pure-Dent® powder into a jar following by adding appropriate amounts of the powder of Example 3, Example 5, and Example 7, following by mixing the powders well.

The body deodorant powders samples were applied on the forearm. The ability of the products to provide multiple fragrance burst was evaluated one hour and after 6 hours following application of the product by misting the area with water. The product comprising the controlled release system of the present invention was found to provide high impact fragrance burst upon wetting the forearm area with water whereas the control samples comprising the neat oil or the encapsulated nonbursting fragrance did not.

| | Response to Moisture | |
|---|---|---|
| | Evaluated after | Evaluated after |
| | 1 Hour | 6 Hours |
| Neat Fragrance Oil (Example 1) | 4 | 3 |
| Encapsulated Fragrance (Example 3) | 7 | 5 |
| Encapsulated Fragrance (Example 4) | 4 | 2 |
| Encapsulated Fragrance (Example 5) | 6 | 6 |
| Encapsulated Fragrance (Example 6) | 3 | 2 |
| Encapsulated Fragrance (Example 7) | 8 | 5 |
| Encapsulated Fragrance (Example 8) | 4 | 3 |

What is claimed is:

1. A controlled release composition comprising:
a plurality of moisture sensitive micro-spheres, said moisture sensitive micro-spheres are formed of a moisture sensitive matrix material; and
a fragrance composition encapsulated in each of said micro-spheres, said fragrance composition comprises menthol and optionally one or more other fragrance ingredients having a ClogP of less than or equal to about 4.0,
wherein said fragrance composition is prevented from being released from said micro-sphere and is released from each of said micro-spheres in a fragrance burst upon contact of each of said micro-spheres with moisture.

2. The composition of claim 1 wherein said fragrance composition has a boiling point of less than or equal to about 300° C.

3. The composition of claim 1 wherein said fragrance composition further comprises less than about 15 percent by weight of a nonbursting fragrance ingredient having a ClogP greater than about 4.0 and a boiling point greater than about 300° C.

4. The composition according to claim 1 wherein said moisture sensitive matrix material is selected from the group consisting of polyvinyl pyrrolidone, water soluble cellulose, polyvinyl alcohol, ethylene maleic anhydride copolymer, methyl vinyl ether maleic anhydride copolymer, polyethylene oxides, polyamide, polyester, copolymers or homopolymers of acrylic acid, polyacrylic acid, polystyrene acrylic acid copolymer, starch derivatives, polyvinyl alcohol, polysaccharide, hydrocolloid, natural gum, protein, and mixtures thereof.

5. The composition of claim 1 wherein said moisture sensitive matrix material is formed of polyvinyl alcohol in an amount of about 1% to about 80% by weight of said moisture sensitive matrix material.

6. The composition of claim 5 wherein said polyvinyl alcohol has a degree of hydrolysis from about 75% to about 99%.

7. The composition of claim 1 wherein said fragrance composition is present in about 0.01% to about 80% by weight of said micro-spheres.

8. The composition of claim 1 wherein each of said micro-spheres has a size of from about 0.5 to about 300 microns.

9. The composition according to claim 1 wherein each of said micro-spheres has an average size of about 0.01 to about 5 microns.

10. A anhydrous controlled release composition for use in personal care comprising:
a plurality of moisture sensitive micro-spheres, said moisture sensitive micro-spheres are formed of a moisture sensitive matrix material; and
a fragrance composition encapsulated in each of said micro-spheres, said fragrance composition comprises menthol and optionally one or more fragrance ingredients having a ClogP of less than or equal to about 4.0,
wherein said one or more fragrance composition is released from each of said microspheres in a fragrance burst upon contact of each of said micro-spheres with moisture.

11. A spray product comprising the composition of claim 10.

12. The spray product of claim 10 wherein said fragrance burst results in increasing said fragrance composition in headspace of a proximate environment of said composition.

13. The spray product of claim 10 wherein said spray product is a deodorant, antiperspirant, body spray, foot spray, hygiene spray, feminine napkin spray or undergarment spray.

14. A powder product comprising the composition of claim 10.

15. The powder product of claim 14 wherein said fragrance burst results in increasing said fragrance composition in headspace of a proximate environment of said composition.

16. The powder product of claim 15 further comprising an active ingredient encapsulated in at least one of said micro-spheres, said one or more agents selected from the group consisting of: anti-oxidants; free radical scavengers; moisturizers; depigmentation agents; reflectants; humectants; antimicrobial agents; antibacterial agents; allergy inhibitors; anti-acne agents; anti-aging agents; anti-wrinkling agents, antiseptics; analgesics; keratolytic agents; anti-inflammatory agents; fresheners; healing agents; anti infective agents; inflammation inhibitors; wound healing promoters; peptides, polypeptides; proteins; deodorants; antiperspirants; skin emollients; skin moisturizers; tanning agents; skin lightening agents; antifungals; depilating agents; counterirritants; poison ivy agents; poison oak agents; burn products; make-up preparations; vitamins; amino acids and their derivatives; herbal extracts; cooling agents; heating agents; skin conditioners; chelating agents; cell turnover enhancers; coloring agents; sunscreens; nourishing agents; moisture absorbers; sebum absorbers; and skin penetration enhancers.

17. The powder product of claim 14 wherein said powder product is a deodorant body powder.

18. A stick product or device comprising the composition of claim 10.

19. The stick product or device of claim 18 wherein said fragrance burst results in increasing said one or more fragrance ingredients in headspace of a proximate environment of said composition.

20. The stick product or device of claim 18 wherein said first active agent comprises one or more agents selected from the group consisting of: anti-oxidants; free radical scavengers; moisturizers; depigmentation agents; reflectants; humectants; antimicrobial agents; antibacterial agents; allergy inhibitors; anti-acne agents; anti-aging agents; anti-wrinkling agents, antiseptics; analgesics; keratolytic agents; anti-inflammatory agents; fresheners; healing agents; anti infective agents; inflammation inhibitors; wound healing promoters; peptides, polypeptides; proteins; deodorants; antiperspirants; skin emollients; skin moisturizers; tanning agents; skin lightening agents; antifungals; depilating agents; counterirritants; poison ivy agents; poison oak agents; burn products; make-up preparations; vitamins; amino acids and their derivatives; herbal extracts; cooling agents; heating agents; skin conditioners; chelating agents; cell turnover enhancers; coloring agents; sunscreens; nourishing agents; moisture absorbers; sebum absorbers; and skin penetration enhancers.

21. The stick product or device of claim 18 wherein said stick product is a lip balm, lipstick, makeup stick, underarm deodorant stick, underarm antiperspirant stick, underarm deodorant roll-on, underarm antiperspirant roll-on, underarm deodorant smooth-on or underarm antiperspirant smooth-on.

22. An article comprising the composition of claim 1.

23. The article of claim 23 wherein said fragrance burst results in increasing said fragrance composition in headspace of a proximate environment of said composition.

24. The article of claim 23 wherein said article is a soap bar.

25. A method for forming the composition of claim 1 comprising the steps of:
emulsifying said fragrance composition in an aqueous phase and said moisture sensitive matrix material in an aqueous phase to form an emulsion; and
removing moisture from said emulsion to form a dry powder composition.
